(19) United States
(12) Patent Application Publication
(10) Pub. No.: US 2016/0256360 A1
(43) Pub. Date: Sep. 8, 2016

(54) Gastronomy Tube Holder

(71) Applicant: Kevin Boyd Goodwin, Slidell, LA (US)

(72) Inventor: Kevin Boyd Goodwin, Slidell, LA (US)

(21) Appl. No.: 14/639,285

(22) Filed: Mar. 5, 2015

(51) Int. Cl. A61J 15/00; A61M 39/10

(52) U.S. Cl. CPC A61J 15/0061; A61M 39/10; A61J 15/0015

(57) ABSTRACT

A percutaneous endoscopic gastrostomy feeding tube is supported with a lanyard connected to a first connector; wherein the first connector operably supports a PEG tube. The first connector may be attached to one or more additional connectors interposed between the lanyard and the primary connector. One or more of the connectors may be a clamp of the type that is biased to the closed, i.e. grasping configuration, and when manually pinched the clamp is opened. Connectors may include one or more clamps of the type the may be locked in the closed, i.e. clamping, position. Connector may include one or more hooks, loops, or straps.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

This invention relates to a device and method for supporting a percutaneous endoscopic gastrostomy feeding tube.

A percutaneous endoscopic gastrostomy feeding tube (also known as a PEG tube, GT, stomach tube, or feeding tube) allows feeding directly into the gastrointestinal tract to occur by bypassing the mouth and esophagus. The PEG tube extends from an external, proximal end, through the skin and the abdominal wall and into the stomach, where the distal end of the PEG tube is resident. Typically, a tube clamp is positioned between the proximal end of the PEG tube and the abdominal wall to open or shut the PEG tube, as appropriate.

Various connectors and interfaces are used where the PEG tube passes through the skin, and within the abdomen and stomach. The purposes of such connections and interfaces include mechanically stabilizing the PEG tube, and limiting potential irritation and infection of the patient's surgical site. Such connectors and interfaces are well known to those skilled in the art.

A port, also known as an adapter, is attached to the proximal end of the PEG tube. To consume nutrition or medication, a syringe, bag, or other container is attached to the port; the tube clamp is opened; and then the nutrition and/or medication is allowed to flow into and through the port, thence into and through the PEG tube, and finally into the patient's stomach. After feeding, the tube clamp is closed, the food container is detached from the port, and the parts are cleaned, disposed of, or sanitized as appropriate.

Between feedings, the external portion of the PEG tube may be stabilized by various means. Harness and belt devices have been used to hold the distal portion of the PEG tube when not in use. Typical current clinical practice involves taping the proximal end and/or adapter of a PEG tube to the patient's abdomen or chest.

Other methods of stabilizing PEG tubes between feedings are documented in the prior art:

U.S. Pat. No. 7,850,669 B1 (Moore et al.), granted 3 Jul. 2006, discloses a tether removably attached to a feeding tube and configured to secure the feeding tube to a neck or torso of a person receiving the feeding tube.

US 2013/0245557 A1 (Brasington), published 16 Mar. 2012, discloses a medical lanyard for securing a feeding tube to a patient; the lanyard is worn around a patient's neck or clipped to the patient's clothing; the lanyard suspends a feeding tube port connector and the corresponding feeding tube.

Heretofore, means of stabilizing the proximal end and/or adapter of a PEG tube were uncomfortable, complex, subject to failure, unsightly, and/or expensive. There is a need for PEG tube support systems that are comfortable, attractive, simple, failure resistant, and inexpensive.

### BRIEF SUMMARY OF THE INVENTION

The present invention meets the needs presented above by providing a lanyard with a first connector; wherein the first connector operably supports a PEG tube.

In certain embodiments of the present invention, the lanyard is attached to the first connector, and the first connector supports a port of a PEG tube; and the port supports the PEG tube.

Additional embodiments of the present invention include one or more additional connectors interposed between the lanyard and the first connector.

In some embodiments of the present invention, the first connector is a clamp of the type that is biased to the closed, i.e. grasping configuration, and when manually pinched the clamp is opened.

In alternate embodiments of the present invention, the first connector is a clamp of the type the may be locked in the closed, i.e. clamping, position.

In various embodiments of the present invention, the first connector is hook, a loop, or a strap.

### BRIEF DESCRIPTION OF THE SEVERAL VIEWS OF THE DRAWING

FIG. 1 depicts a PEG tube support, showing a partial lanyard (1) directly attached to a connector (3), the connector being attached to a port (7), and the port being attached to a PEG tube (8).

FIG. 2 depicts an alternate embodiment of a PEG tube support, showing a partial lanyard (1) attached to a first connector (4). The first connector is attached to a second connector (9). The second connector is attached to port (7); and the port is attached to PEG tube (8).

FIG. 3 illustrates another embodiment of a PEG tube support, showing a partial lanyard (1) operably attached to the PEG tube (8), via a first connector (5), a second connector (9), and port (7). FIG. 2 also calls out proximal ends of the lanyard (1p, 1p′), and a distal end of the lanyard (1d).

FIG. 4 shows a PEG tube support including a closed loop lanyard (2), with proximal (2p ) and distal (2d ) ends, attached to a first connector (5). The first connector is attached to a second connector (9); and the second connector is attached to port (7), the port being attached to PEG tube (8). The PEG tube includes an in-line shut-off valve (10).

FIG. 5 presents a PEG tube support including a closed loop lanyard (2), attached to first connector (6). The first connector is attached to a second connector (10); and the second connector is attached to a third connector (3). The third connector being attached to a port (not shown), the port being attached to PEG tube (not shown).

### DETAILED DESCRIPTION OF THE INVENTION

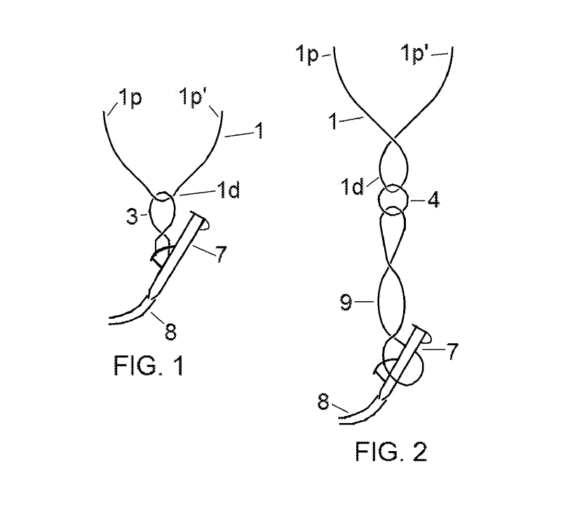

The PEG tube connector includes a lanyard. Typically the lanyard is worn around the neck of the patient. The lanyard may be a single strap, a bifurcated strap, or a closed loop. FIGS. 1-5 illustrate lanyards (1, 2). The lanyard may include one or more clips (not shown) to secure it to clothing or other items. The lanyard includes a proximal end and a distal end.

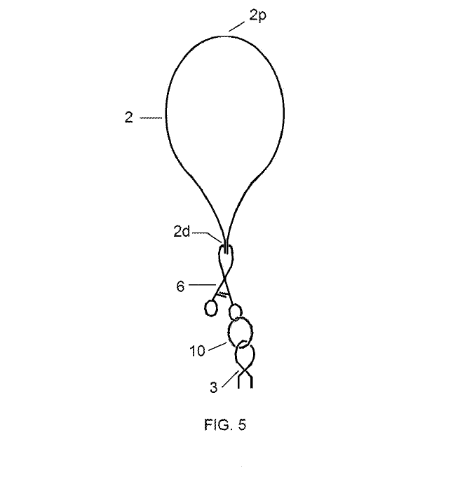

In FIGS. 1-3 partial lanyards (1) are shown with respective proximal ends (1p, 1p′) and distal ends (1d ). In FIGS. 4 and 5 closed loop lanyards (2) are shown with respective proximal ends (2p ) and respective distal ends (2d ). In use, the proximal end(s) of the lanyard is typically worn at the back of the neck; and the distal end of the lanyard typically rest on the chest of the patient.

Partial lanyards may comprise two separate proximal ends. For the purpose of this disclosure, the terms “separate ends” and “separable ends” are used interchangeably, both terms indicate that the ends can be manufactured as a unitary piece, or separate pieces; and the ends include means to separate, unite, or reunite the ends. The separable ends may be connected using hooks and loops, including microhooks, buttons, cohesive or adhesive surfaces, with corresponding fastening means on each of the separable ends. Alternatively, the ends may be united tying a knot. In some embodiments of the invention, the separable ends of the lanyard are manufactured as a unitary that is weakened with slits, cuts, or embossments; and configured to be separated. Partial lanyards are typically place around the neck of the patient and then the proximal ends are connected. Closed loop lanyards typically are slipped over the patient's neck before using the PEG tube support.

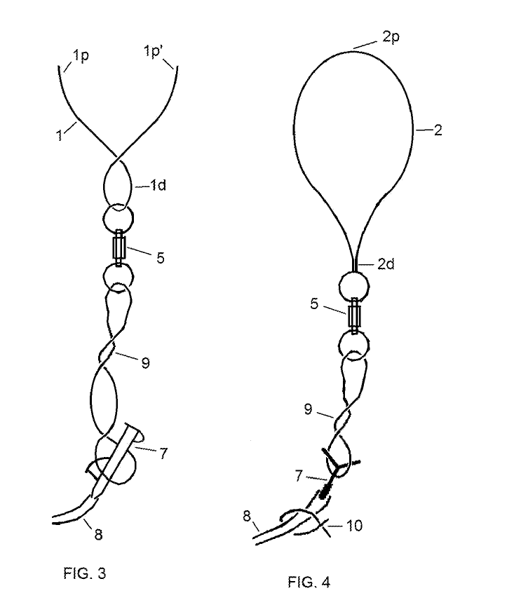

Lanyards may be stitched, stapled, tied, or adhesively bonded or otherwise joined to form a distal end. Alternatively, the lanyard may be directly formed into the desired shape; for example by extruding or molding. As shown in FIG. 1, the distal end (1d ) of the lanyard may simply be a portion of the loop that goes about the neck or the patient. As shown in FIGS. 2 and 3, the distal end (1d ) of the lanyard may include a twist, forming a second loop. As shown in FIGS. 4 and 5, the distal end of the lanyard may include a tail portion (2d ), i.e. a unitary strap. The lanyard may be made of an elastic strap; a cord, a rope, a woven or non-woven fabric; a monofilament fiber; a knitted, crocheted, braided or twisted yarn; or a chain, or combinations thereof. The lanyard may be comprised of natural materials, such as cotton, wool, rubbers, or silk, or synthetic polymers such as polyester, nylon, rubbers, polyolefins, polyethylene, polypropylene, or polytetrafluoroethylene or metals.

The lanyard may include a length adjustment mechanism to adjust the length of the lanyard (not illustrated). For example slide adjusters, such as those used for suspenders or web belts can be used. In a slide adjuster, one portion of the lanyard is fixed to the adjuster, and the other portion of the adjuster can be moved in or out until the desired length is achieved, then the adjuster is locked. Such slide adjusters have been widely used for garments for many years. Alternative length adjustment mechanisms include draw strings.

In addition to providing the appropriate softness, elasticity, moisture absorption or wicking properties, and strength needed for the application; the lanyard may be ornamental or provide indicia. The lanyard may be decorative, colored or festooned with ornamental elements or precious or semi-precious stones. Additionally, the lanyard may illuminated or include designs or identifying marks. The lanyard may comprise a tarnish-resistant or precious metal, such as titanium, gold, platinum, or silver. In other embodiments the lanyard may be transparent, translucent, or colored to match the wearer's clothing. Furthermore, the lanyard may be configured to appear as a collar, or other article of clothing.

The lanyard is affixed to a first connector (3, 4, 5, 6).
In FIG. 1 connector (3) connects directly to port (7); and port (7) connects to PEG tube (8).
In FIG. 2, first connector (4) connects to second connector (9); second connector (9) connects to port (7); and port (7) connects to PEG tube (8).
In FIG. 3, first connector (5) connects to second connector (9); second connector (9) connects to port (7); and port (7) connects to PEG tube (8).
In FIG. 4, first connector (5) connects to second connector (9); second connector (9) connects to port (7); and port (7) connects to PEG tube (8).
In FIG. 5, first connector (6) connects to second connector (10); second connector (10) connects to third connector (3); third connector (3) connects to port and the port connects to PEG tube.

Each connector, may comprise a solid ring, a spilt ring, a ring with a gap, a carabiner, a loop, a clamp, a swivel mechanism, a hook, a chain, a cable tie, tie-wrap, hose tie, zap-strap, zip tie, a strap, a band, a wire, or other connector, or combinations thereof. Swivel mechanisms allow free rotation around at least one axis; for example swivel mechanisms include barrel and ball swivels. Swivel mechanisms are widely available and have been used as components for medical devices, jewelry, and fishing lures for many years. The connectors may be made of metal, plastic, or other synthetic or natural materials. The connectors may include a hook and loop fastener system, or a microhook fastener system such as the types sold by 3M Corporation (St. Paul, Minn.) and Velcro USA (Manchester, N.H.), and their equivalents. The lanyard may include a portion of the hook and loop fastener system to facilitate attachment to itself or to a connector. The figures, and the discussion below, provide examples of various embodiments of the invention.
FIG. 1 shows the first connector (3) as a medical tube clamp, with lanyard (1) looped through the clamp; and the clamp grasping a portion of port (7).
FIG. 2 shows the first connector 42) as a ring, with lanyard (1) looped through the ring; and the ring passing through second connector (9).
FIGS. 3 and 4 show the first connector (5) as a swivel mechanism.
FIG. 5 shows the first connector (6) as a locking forceps.
FIGS. 2-4 show a second connector (9) as a twisted band looped through the first connector (4,5); and the twisted band tied to port (7).
FIG. 5 shows a second connector (10) as a chain, looped through a finger hole in the first connector (6), i.e. locking forceps; and the chain further looped through a third connector (3). Third connector (3) is a medical tube clamp. The chain loops through the clamp, and the clamp grasps a port.
   The various embodiments of the invention disclosed above may be combined in sundry manners, as would be obvious to a person having ordinary skill in the art

In various embodiments of the invention, at least one connector is a strap, band, chain, or other material that forms a loop. The loop and may be a single loop, or it may be twisted to form two or three or more loops.

A connector may include a gap or slit, configured to corresponding engage a lanyard, a port, or another connector. The gap or slit may be left open and positioned so that the corresponding components do not slip through the gap. Alternatively the gap or slit may be closed using fasteners, such as jewelry clasps, or bonded, using adhesives, staples, heat fusing, soldering, sonic fusing, micro-hook fasteners, and the like.

A connector strap may be a metallic color, such as silver, gold, or copper. Alternatively, a connector may be white, transparent, or various colors. The colors may be achieved by leaving the material of the strap in its natural state, or by coating or impregnating with various colorants, as known in the art.

The terms “connects”, “grasps”, “engages”, “clamps” and their derivatives, refer to a lanyard, connector, port, or PEG tube, holding onto another component, regardless of whether there is a pinch grip or an interlocking or interpenetration of links or loops.

In some embodiments of the invention, a clamp that locks in the closed position is used as a connector. For example, a locking hemostat, or a ratchet tubing clamp, a locking clamp (e.g. Vice-Grip(R) clamp), or C-clamp may function as a connector. In some embodiments of the invention, a clamp that is spring loaded is used as a connector.

The various embodiments of the invention disclosed above may be combined in various manners, as would be obvious to a person having ordinary skill in the art. Furthermore the descriptions of various embodiments of the invention are to be considered exemplary. The embodiments of the invention are not to be considered as limiting the scope of the invention as described in the appended claims. All equivalent relationships to those illustrated in the drawings and described in the specification are intended to be encompassed by the present invention.

I claim:

1. A device for supporting a percutaneous endoscopic gastrostomy feeding tube (PEG tube) on a patient comprising:
a lanyard worn about the neck of the patient, the lanyard comprising a loop, with a proximal portion of the loop positioned near the back of the neck of the patient, and a distal portion of the loop worn on or near the chest of the patient; and
a connector attached to the lanyard; wherein the connector is configured to engage a portion of the PEG tube or a port attached to the PEG tube.

2. The device of claim 1, wherein the distal portion of the lanyard comprises a tail.

3. The device of claim 1, wherein the lanyard comprises a length adjustment mechanism.

4. The device of claim 1, wherein the connector comprises a swivel mechanism.

5. The device of claim 1, wherein the distal portion of the lanyard comprises a loop, and the proximal portion of the lanyard comprises separable pieces.

6. The device of claim 1, wherein:
the distal portion of the lanyard comprises a tail portion;
the lanyard comprises a length adjustment mechanism; and
the connector comprises a swivel mechanism.

7. A device for supporting a PEG tube on a patient comprising:
a lanyard worn about the neck of the patient, the lanyard comprising a loop, with a proximal portion of the loop positioned near the back of the neck of the patient, and a distal portion of the loop worn on or near the chest of the patient;
a first connector attached to the lanyard and
a second connector attached to the first connector, wherein the second connector is configured to grasp a portion of the PEG tube or a port attached to the PEG tube.

8. The device of claim 7, wherein the distal portion of the lanyard comprises a tail.

9. The device of claim 7, wherein the lanyard comprises a length adjustment mechanism.

10. The device of claim 7, wherein the first connector comprises a swivel mechanism.

11. The device of claim 7, wherein the distal portion of the lanyard comprises a loop, and the proximal portion of the lanyard comprises separable pieces.

12. The device of claim 7, wherein:
the distal portion of the lanyard comprises a tail portion;
the lanyard comprises a length adjustment mechanism;
at least one of the connectors comprises a swivel mechanism; and
the second connector comprises a clamp.

13. The device of claim 7, wherein
at least one of the connectors comprises a swivel mechanism;
at least one of the connectors comprises a split ring, a ring with a gap, a clamp, a hook, or a loop, configured to engage a corresponding portion of the other connector;
the second connector comprises a clamp to grasp a portion of the PEG tube or a port attached to the PEG tube.

14. A device for supporting a PEG tube on a patient comprising:
a lanyard worn about the neck of the patient, the lanyard comprising a loop, with a proximal portion of the loop positioned near the back of the neck of the patient, and a distal portion of the loop worn on or near the chest of the patient;
a first connector attached to the lanyard;
a second connector attached to the first connector; and
a third connector attached to the second connector, wherein the third connector is configured to grasp a portion of a PEG tube or a port attached to the PEG tube.

15. The device of claim 14, wherein the third connector comprises a clamp configured to grasp a port attached to the port or the PEG tube.

16. The device of claim 14, wherein the lanyard comprises a length adjustment.

17. The device of claim 14, wherein at least one of the first connector, the second connector, and the third connector is a swivel mechanism.

18. The device of claim 17, wherein the distal portion of the lanyard comprises a loop, and the proximal portion of the lanyard comprises separate ends; wherein the ends are configured to unite using any combination of hooks, loops, adhesive, and cohesive surfaces.

19. The device of claim 17, wherein at least one of the connectors comprises a split ring, a ring with a gap, a clamp, a hook, or a loop, configured to engage a corresponding portion of an adjacent connector.

20. The device of claim 19, wherein the lanyard comprises an elastic material.
